(19) United States
(12) Patent Application Publication
(10) Pub. No.: US 2020/0161001 A1
(43) Pub. Date: May 21, 2020

(54) Personal Data Analytics System

(71) Applicant: Neurostim Technologies LLC, Waltham, MA (US)

(72) Inventors: Hoo-min D. TOONG, Cambridge, MA (US); Paul ALBRECHT, Bedford, MA (US); Paul E. GRIMSHAW, Acton, MA (US); Tiejun ZHANG, West Roxbury, MA (US)

(21) Appl. No.: 16/689,577

(22) Filed: Nov. 20, 2019

Related U.S. Application Data
(60) Provisional application No. 62/769,975, filed on Nov. 20, 2018.

(51) Int. Cl. G16H 50/30; G16H 10/60; A61B 5/00

(52) U.S. Cl. CPC G16H 50/30; G16H 10/60; A61B 5/746; A61B 5/7435; A61B 5/6833; A61B 5/7282

(57) ABSTRACT

Example inventions evaluate a health of a user. Example inventions receive, from a plurality of disparate data sources, health data corresponding to the user, the plurality of disparate data sources including two or more of biometric data, activity monitoring, or an electronic health record. Example inventions correlate the disparate data sources onto a common timeline. Example inventions generate a user interface that displays the correlated data sources simultaneously with the common timeline.

### CROSS REFERENCE TO RELATED APPLICATIONS

This application claims priority to U.S. Provisional Patent Application Ser. No. 62/769,975, filed on Nov. 20, 2018, disclosure of which is hereby incorporated by reference.

### FIELD

Example inventions are directed generally to a computer system, and in particular to a computer system for monitoring biometric data and correlating the biometric data with health-related information for analytics.

### BACKGROUND INFORMATION

There has recently been a proliferation of devices, many that are wearable, that generate health-related data, including biometric data, for the user. For example, devices track a user's heartbeat, blood pressure, number of steps taken or other activity, electrocardiogram (“EKG”) information, etc. However, in general, this health data, when it comes from separate disparate devices or other sources, has not easily been aggregated in a way to be useful to a user and/or health provider.

Generally, there is a need to have the ability to view health history data and how it correlates to other information in the health field, with the goal of modifying an individual's behaviors to improve his or her health, or the health of a person under his or her care. Users may include entities in a variety of situations seeking to view individuals' health data and correlate that data to other information, such as a person viewing and correlating their own health data to their health history and to aggregated health data across a population, and medical professionals, such as doctors, nurses, caregivers, hospitals; researchers in academia, government, industry and insurers.

### BRIEF DESCRIPTION OF THE DRAWINGS

FIG. 1 illustrates a personal data analytics system in accordance with example inventions.

FIG. 2 illustrates a personal data analytics system in accordance with example inventions.

FIG. 3 illustrates a personal data analytics system in accordance with example inventions.

FIG. 4 illustrates a personal data analytics system in accordance with example inventions.

FIG. 5 is a flowchart of reporting functionality in accordance with example inventions.

FIG. 6A is a user interface (“UI”) for displaying generated information in accordance to example inventions.

FIG. 6B is a UI for displaying generated information in accordance to example inventions.

FIG. 7 illustrates an overall system that combines data sources and performs data analysis in accordance to example inventions.

FIG. 8 is a block diagram of a computer server/system in accordance with examples of the present invention.

FIG. 9 is a UI for displaying generated information, including correlations and alerts, in accordance to example inventions.

FIG. 10 is a UI for displaying generated information in accordance to example inventions.

FIG. 11 is a flow diagram of the personal data analytics module of FIG. 8 when generating personal data analytics in accordance to example embodiments.

### DETAILED DESCRIPTION

An individual/user has a need to learn how their health can be improved by monitoring their own biometric data over time, and how the biometric data correlates to pharmaceutical, medical, and other data specifics contained in the person's health record, and how the data correlates to larger data sets from populations. Examples of the invention are directed to displaying a user's health data across a span of time, (referred to as “Biometric Monitoring” data), with markers to indicate how variations in the health data correlate to specifics from a wider range of data sets, such as pharmaceutical data, medical diagnostic data, or a user's own life data (referred to as a “user health record”). Further, example inventions generate a user interface that displays the disparate sources of information onto a common timeline, which generates and facilitates further analytics regarding the combination of information.

There is further a need for correlation of disparate data sources when a person or organization wants to learn how an individual's or a group's health can be improved. Factors that promote health improvement can be assessed by correlating collected health data with larger data sets, including displaying the disparate data along a common timeline. This information can be used to guide therapies and healthy behaviors. The present invention is directed to a system and method to display a user's health data across a span of time and a common timeline with other information, as described above. Example inventions may also be used to display health data for an individual or aggregate data across a population of individuals.

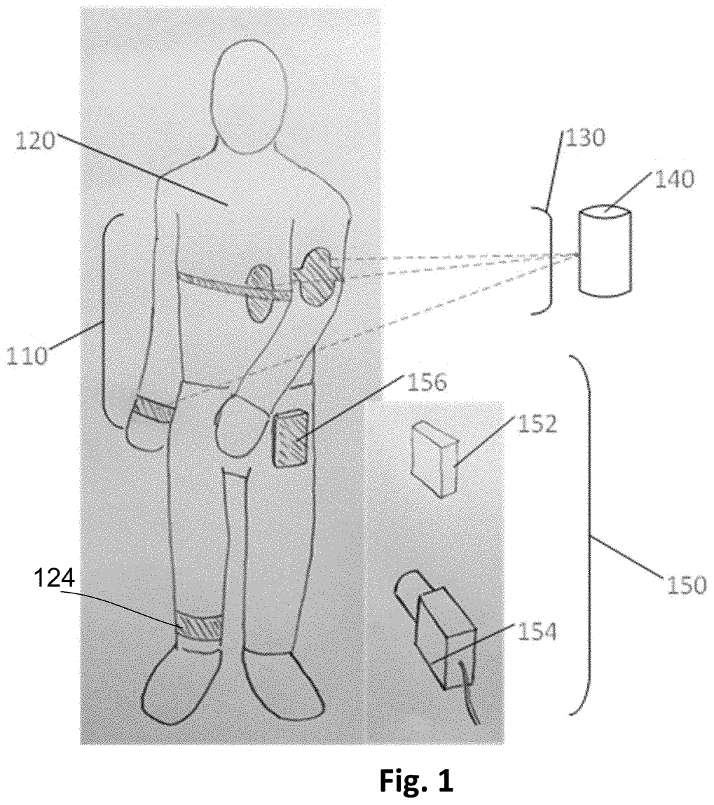

FIG. 1 illustrates a personal data analytics system in accordance with example inventions. In FIG. 1, one or more health devices 110 monitor a user 120 and collects user biometric data 130, which is sent to a user data store or database 140. Data store 140 also collects data from one or more user monitoring devices 150, such as a Global Positioning System (“GPS”) device 152, a camera 154 and a smartphone 156. Health devices 110 may include electrocardiogram (“ECG”) monitors, electroencephalogram (“EEG”) monitors, fitness devices, blood pressure monitors, activity monitors, etc.

In some examples, health devices 110 include one or more smart patches 124 worn by user 120. In examples, a “smart” patch 124 is attached to the medial malleolus of user 120 on the right or left ankle of user 120 in accordance to examples.

The placement of each patch 124 is designed to cause electrical stimuli to activate the tibial nerve of user 120 in one example to alleviate overactive bladder (“OAB”) symptoms. The term “smart”, in general, refers to the use of memory and logic components and instructions, and may also include electronic components for communications, to generate some or all of the functionality disclosed herein.

Patch 124 can be any type of device that can be fixedly attached to user 120 and includes a processor/controller and instructions that are executed by the processor, or a hardware implementation without software instructions, and communication elements to provide communications with a controller (e.g., smartphone 156 or a fob) in some examples. Patch 124 can also include additional components that provide topical nerve stimulation on user 120 to provide benefits to user 120, including bladder management for an overactive bladder, such as electrodes, sensors, a battery, adhesive, a control unit, an electronic integrated package, stimulators, etc.

Patch 124 in one example can include a flexible substrate, a malleable dermis conforming bottom surface of the substrate including adhesive and adapted to contact the dermis, a flexible top outer surface of the substrate approximately parallel to the bottom surface, one or more electrodes positioned on the patch proximal to the bottom surface and located beneath the top outer surface and directly contacting the flexible substrate, electronic circuitry embedded in the patch and located beneath the top outer surface and integrated as a system on a chip that is directly contacting the flexible substrate, the electronic circuitry integrated as the system on the chip and including an electrical signal generator integral to the malleable dermis conforming bottom surface configured to electrically activate the one or more electrodes, a signal activator coupled to the electrical signal generator, a nerve stimulation sensor that provides feedback in response to a stimulation of one or more nerves, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to activate/stimulate one or more nerves of a user wearing patch 124 at least at one location proximate to patch 124. Additional details of examples of patch 124 are disclosed in U.S. Pat. No. 10,016,600, entitled “Topical Neurological Stimulation”, the disclosure of which is hereby incorporated by reference.

In some examples, health monitoring devices 110, such as patch 124, can send and receive data from a controller, and send the data on to a cloud service. In some examples, patch 124 stimulates the tibial nerve of user 120 at the direction of a doctor/caregiver via direct or remote activation to elicit a suppressive nerve response, which, in turn, suppresses the urination impulse.

In some examples, the system measures the state of the patient's bladder to determine the degree of urgency in voiding the bladder. In some examples, the system uses ultrasound to measure the state of the bladder.

In some examples, the system may measure other biometric attributes of user 120 to determine the degree of urgency in voiding the bladder. Examples of these measurements may be a clenching of abdominal muscles, or restlessness during sleep, or the shape or opacity of the bladder when imaged.

Biometrics refers to body measurements and calculations and metrics related to human characteristics. Biometric identifiers are the distinctive, measurable characteristics used to label and describe individuals and include physiological and behavioral characteristics. Physiological characteristics are related to the shape of the body. Examples include veins, face recognition, DNA, palm print, hand geometry, iris recognition, retina and odor/scent. Behavioral characteristics are related to the pattern of behavior of a person, including typing rhythm, gait, and voice.

In some examples, health monitoring devices 110 include a device for determining when an OAB event has occurred. For example, a moisture detection element in a smart diaper or other garment worn by user 120 can perform analysis to determine an OAB event, and the time of the event, and output this data to the system. In another example, the monitoring of the instances that patch 124 is activated by the user to stimulate the nerves provides an indication of an increase or decrease of OAB conditions and the urge to urinate.

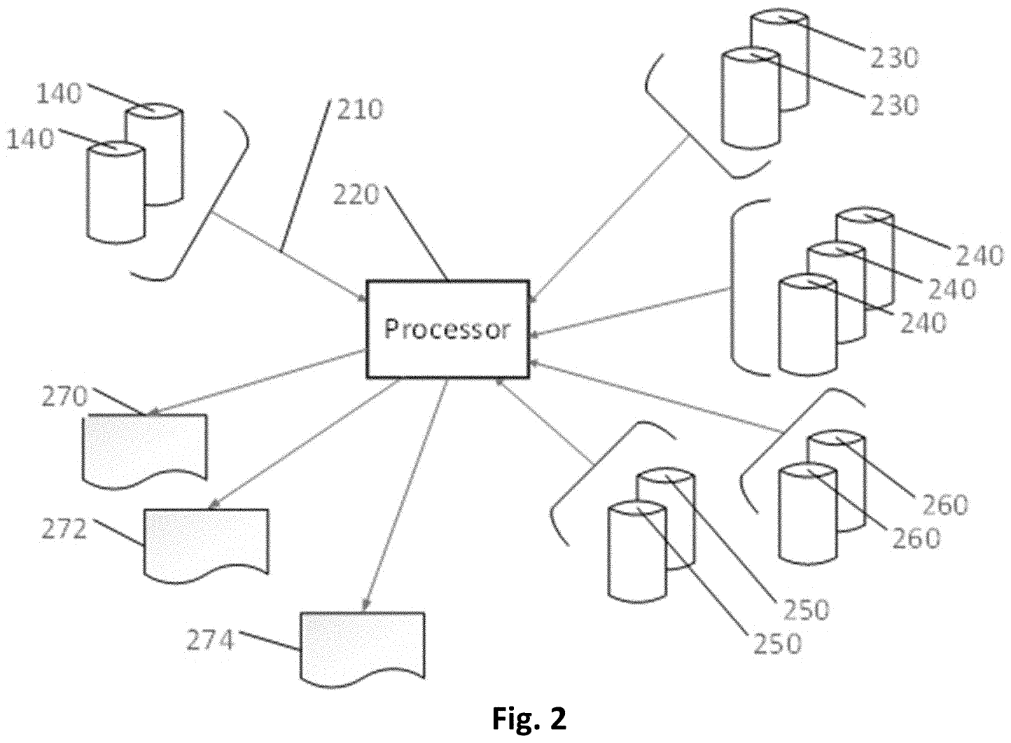

FIG. 2 illustrates a personal data analytics system in accordance with example inventions. In FIG. 2, user data stores 140 provides user data 210 to one or more processors/servers 220. Processor 220 further accesses at least one of a user health record database 230, a drug database 240, a population database 250, or a genetic database 260. Processor 220 analyzes these data sources to create at least one of a user report 270, a professional report 272, or a caregiver report 274.

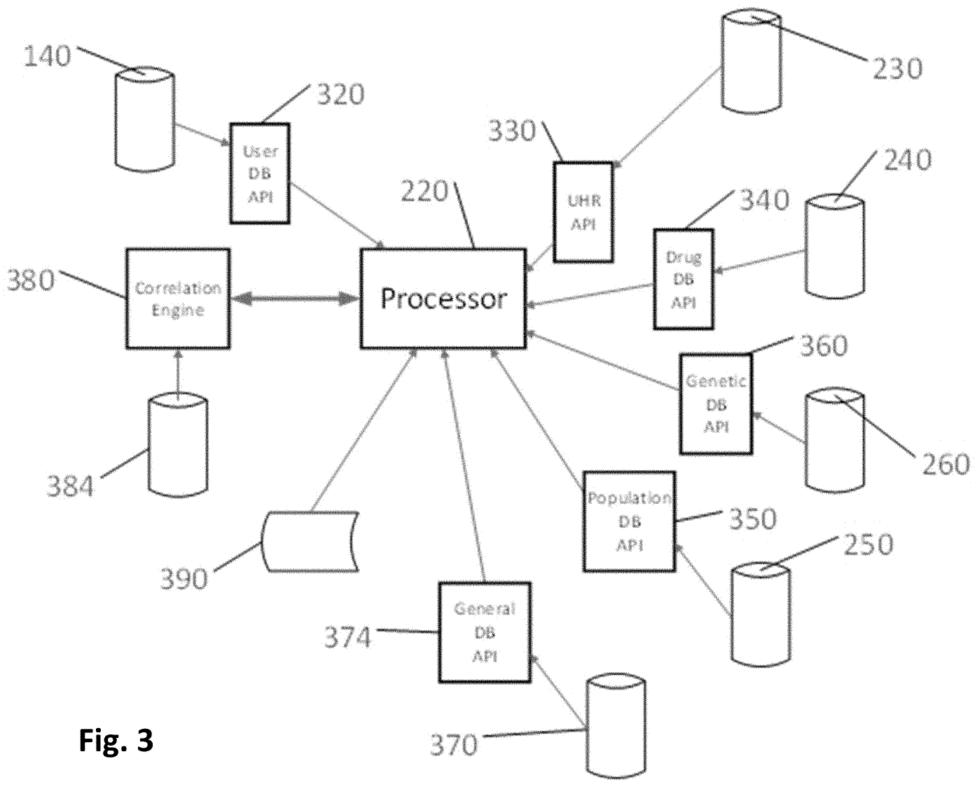

FIG. 3 illustrates a personal data analytics system in accordance with example inventions. In FIG. 3, individual access processes are shown as interfaces between the data stores of FIG. 2 and processor 220. User health record database 230 is accessed through an UHR API 330, making specific user health record data available in a format understandable to processor 220. Similarly, drug database 240 is accessed through a Drug DB API 340; population database 250 is accessed through a Population DB API 350; genetic database 260 is accessed through a Genetic DB API 360; and accesses to other data 370 are made through either specific or general APIs 374. Processor 220 correlates these data streams using a correlation engine 380, which is provided with access to a medical analysis database 384. Correlation engine 380 converts data from various sources into a common format to facilitate data translation and use in analysis and data presentation, including displaying the data on a common timeline. In addition to medical analysis database 384, processor 220 accepts inputs from professional responses 390.

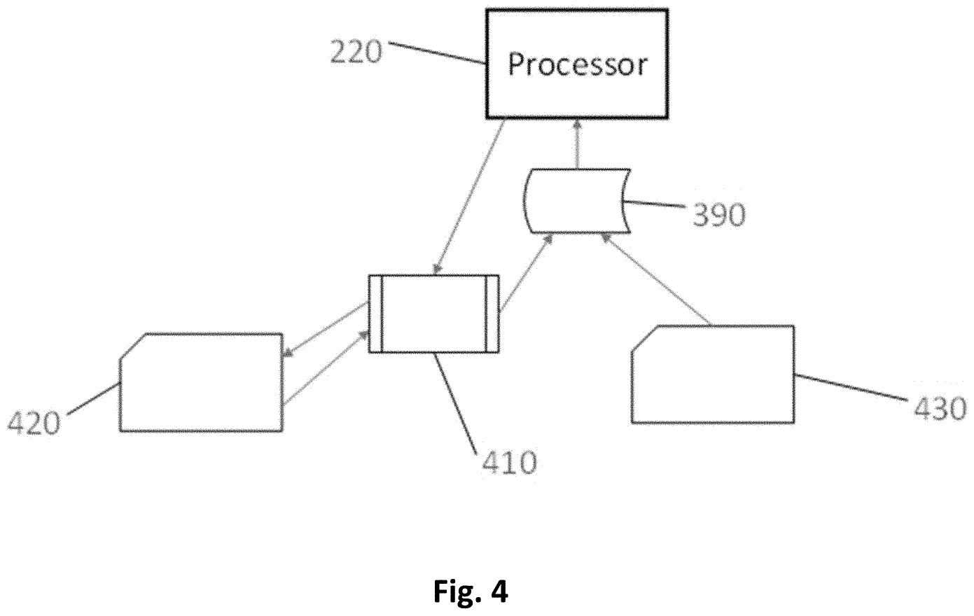

FIG. 4 illustrates a personal data analytics system in accordance with example inventions. In FIG. 4, professional responses 390 are generated from professional input devices 410, which are provided with input questionnaire 420, based on access to processor/server 220, which feeds data into questionnaire 420, and with input forms 430, which medical professionals use to input data for particular medical situations. Professional responses 390 may also occur when the professional, such as a physician, nurse, caregiver, etc., studies the various data sources, either before or after the work of correlation engine 380.

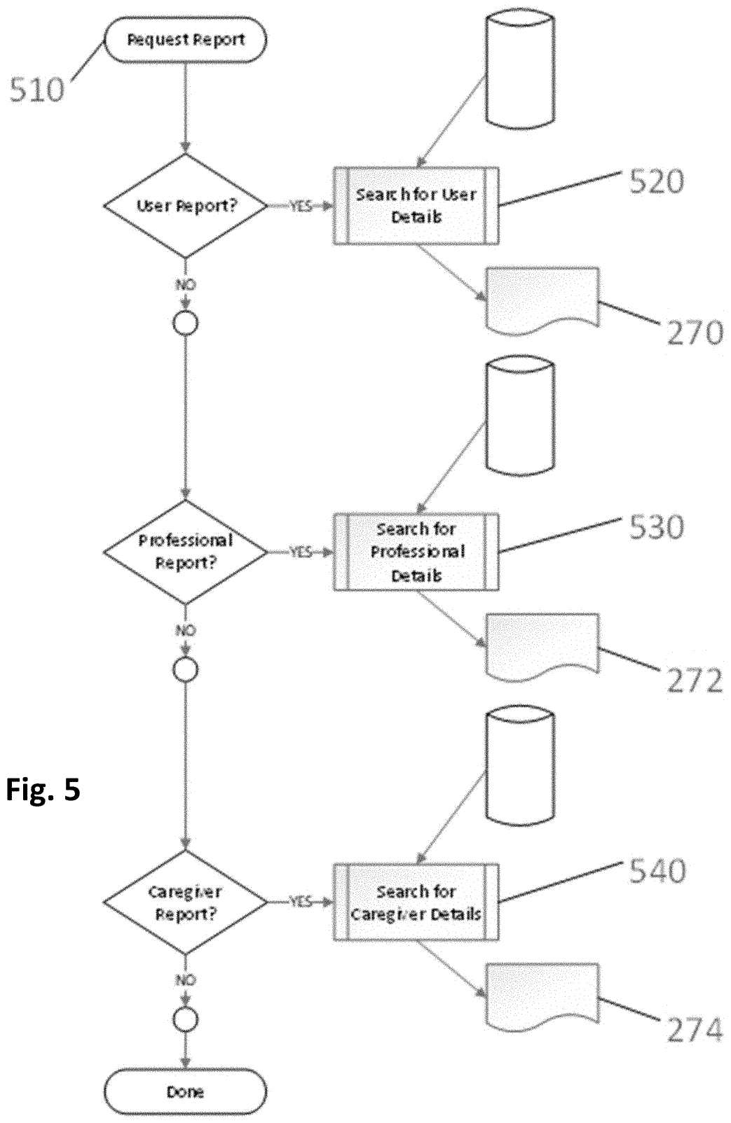

FIG. 5 is a flowchart of reporting functionality in accordance with example inventions. FIG. 5 illustrates a process of creating user report 270 using a user report process 520, and professional report 272 using a professional report process 530, and caregiver report 274 using a caregiver report process 540. Each of the processes draws data from the appropriate databases, and outputs a report.

Referring again to FIG. 1, recent advances in technology have made available to users a variety of devices and systems that can function as health monitoring devices 110 for measuring one or more biometric data type, such as step count, pulse rate, blood pressure, respiration rate, hydration, blood sugar levels, body mass index, etc. Many of these devices provide means for displaying the data collected on the device to the user, with the help of a computer and display or on the device itself. In some cases, the data may be displayed as a data series, across a span of time, the time range being part of the display, such as in hours, days, weeks. Some of these display systems may also show limit lines, such as the recommended minimum or maximum value, or both, for the specific biometric parameter being shown, such as maximum or minimum blood pressure or pulse rate.

The user may discern from the displayed data how his or her health varies across time. This discernment is limited inasmuch as the displayed raw data is not correlated to a variety of underlying mechanisms which affect the user's health. For example, the display of multiple biometric data types, aligned across the time span of the display, may also help the user to understand why their biometric data is varying or is not in the optimum range between recommended minimum and maximum values.

Example inventions can combine biometric data with other data types, such as electronic health records (“EHR”s), using correlations derived from medical information or from statistical analysis of the data or from machine learning or artificial intelligence analysis or from human analysis. This is the work of correlation engine 380 of FIG. 3, which may employ one or more of these data analysis techniques.

A user may better understand the data and glean pertinent information presented in user report 270 because the presentation of the data is designed by user report process 520. Similarly, professionals may derive more value from the data presented in professional report 272 because the presentation of the data is designed by professional report process 530 and caregivers may better understand and take action from the data presented in caregiver report 274 because of the selected formats and content by caregiver report process 540.

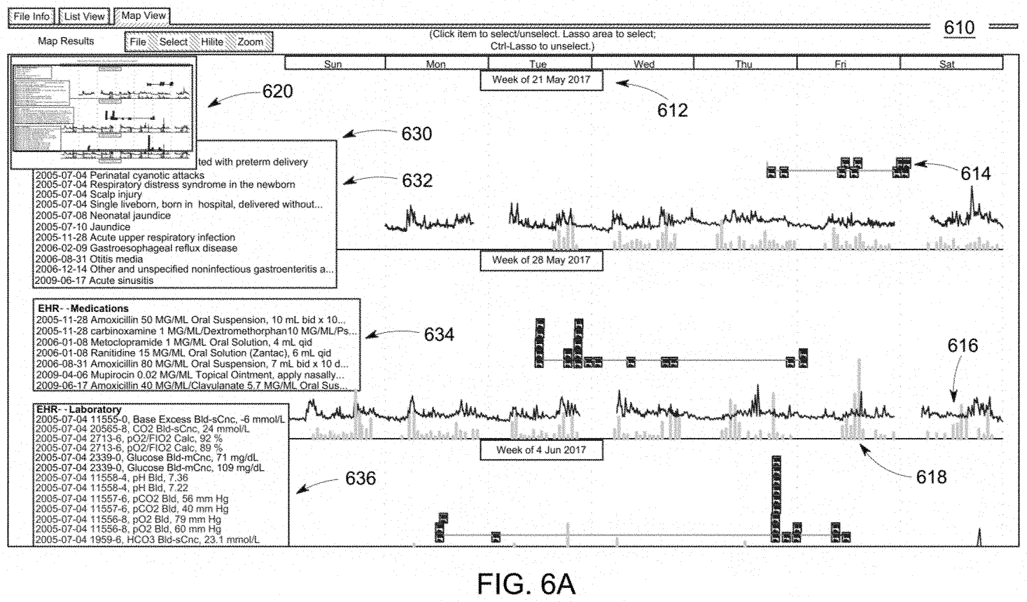

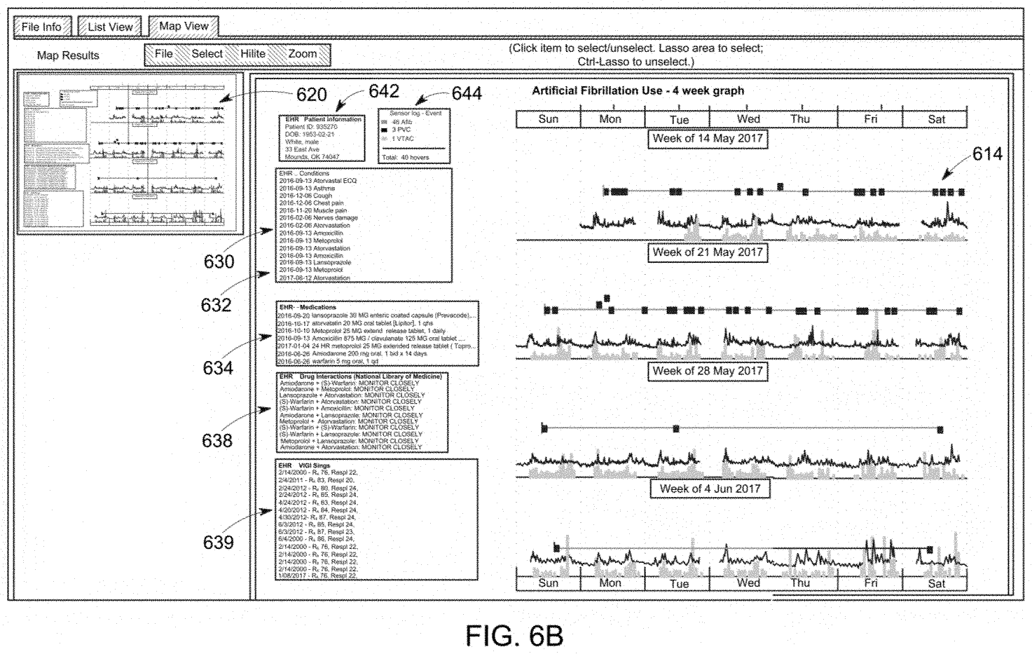

Example inventions generate a user interface that combines data from various sources, to assist the various types of users in drawing conclusions from the information. FIG. 6A is a user interface (“UI”) for displaying generated information in accordance to example inventions. The UI of FIG. 6A includes a user timeline view 610 with timeline labels 612 (i.e., days of the week), event marker icons 614 that indicate OAB events, a line graph trace 616, a bar chart trace 618; navigation window 620; EHR data 630 with EHR event tabular data 632, EHR prescription data 634, and lab test tabular data 636.

FIG. 6B is a UI for displaying generated information in accordance to example inventions. The UI of FIG. 6B includes event marker icons 614 with arrhythmia events; navigation window 620; EHR data 630 with EHR event tabular data 632, EHR prescription data 634, EHR drug interaction data 638, and EHR vital signs data 639; user identification data 642; and a legend 644.

The data in the UIs of FIGS. 6A, 6B is presented on a user interface as timeline 610, the scale of which may be changed from daily to weekly to monthly to yearly view 612, adjusting the level of detail of the user data accordingly. User data is presented according to the type of data, with event data 614 such as OAB/urination events shown as stacked icons; continuous data 616 such as heart rate shown as a continuous line graph; and quantitative, discrete data 618 such as step counts shown as bars along the timeline. Related health data is presented in smaller windows in tabular form 630, including EHR event tabular data 632, such as doctor visits; EHR prescription data 634, such as dosages; EHR lab test tabular data 636, such as historical blood chemistry measurements; EHR drug interaction data 638, including warnings; and EHR vital signs data 639, such as historical blood pressure measurements. The interface is labeled with user identification data 642. The user navigates the user interface with the navigation window 620. Legend 644 aids in navigation. On this combined presentation, the viewer may combine historical data with current data and medical reference data such as drug interaction details to provide the user with directives.

For example, the biometric data showing blood pressure may show a variation with a high value early in each day, excepting Saturdays and Sundays, with a similarly high value late in each weekday. Examples inventions can combine the blood pressure biometric data with a record of the user's movements as collected from their smartphone GPS system on a common timeline, and deduce that the user is moving rapidly during each of the periods when the blood pressure is markedly higher, this movement being further deduced to be in a vehicle. The display of this information across a period of multiple days and weeks, with annotations to highlight the higher blood pressure periods and a note that this is the user's commute time, is all presented to the user on a single UI or printed report. Through the use of example inventions that can include the derivation of a recommendation, the user is able to consider whether to change his or her behavior. In the example, the user may choose to travel to and from work at a different time of day based on the generated data.

In another example, the user provides a diary which records the meals he or she has had, and the foods consumed. Examples inventions have access to this diary data, as well as to the user's prescription drug history through the user's EHR, and the user's biometric data. The pharmaceutical data for the user is made more useful by examples inventions which can access one or more external drug interactions databases. All of these data sources are analyzed and correlated by examples inventions, across a span of time measurements, and examples inventions deduce that the user occasionally consumes foods which adversely affect the effectiveness of one of the user's prescription medications. Further, example inventions deduce that the user is taking a medication at a time of day when the user is driving a vehicle, this medication being prone to cause drowsiness. The user is informed of these relationships through the use of time series displays on a UI or printed report, or as warnings or alerts such as on a smart phone device. Through these means, delivered by examples inventions, the user is able to change his or her behavior by avoiding certain foods, and avoiding the taking of one of the user's medications during driving times.

In another example, the user who has been diagnosed with atrial fibrillation (“AFib”) is instructed by the user's physician to monitor himself or herself using various data-gathering devices. Examples inventions combine the data from those sources into a form and format useful to the medical professional to make further determination for the user, such as modifying the prescription instructions for a particular medication, or limiting user's activities in areas which may aggravate the user's AFib condition, or relaxing restrictions or medications if example inventions reveal that the condition is less serious than was initially diagnosed. The process between the user and the medical professional may proceed as follows: (a) the user reports symptoms to primary care physician (“PCP”): fatigue, light-headedness, reduced ability to exercise; (b) the PCP records an ECG during office visit, and periods of AF are detected, so that the PCP refers the user to a Cardiologist; (c) the Cardiologist has the user wear an ECG monitoring device for a 2-week period to measure baseline AFib burden (% of time in AFib), and has the user also wear a FitBit or similar device to measure physical activity; (d) at a Cardiologist follow up visit, a review of the ECG data reveals overall AFib burden of 21%, such that the Cardiologist prescribes Amiodarone, an anti-arrhythmic medication, along with Warfarin, anti-coagulant, to reduce risk of embolic stroke; (e) the user continues to wear the ECG patch and the FitBit type of device for another 2-week period after starting medication; (f) at a subsequent Cardiologist follow up visit, a review of the ECG and Fitbit type of device data to determine changes in AF burden and physical activity after medication was started reveals that AF burden has been reduced, such that the medication dosages may be adjusted to reduce medication side-effects; and (g) the monitoring process repeated.

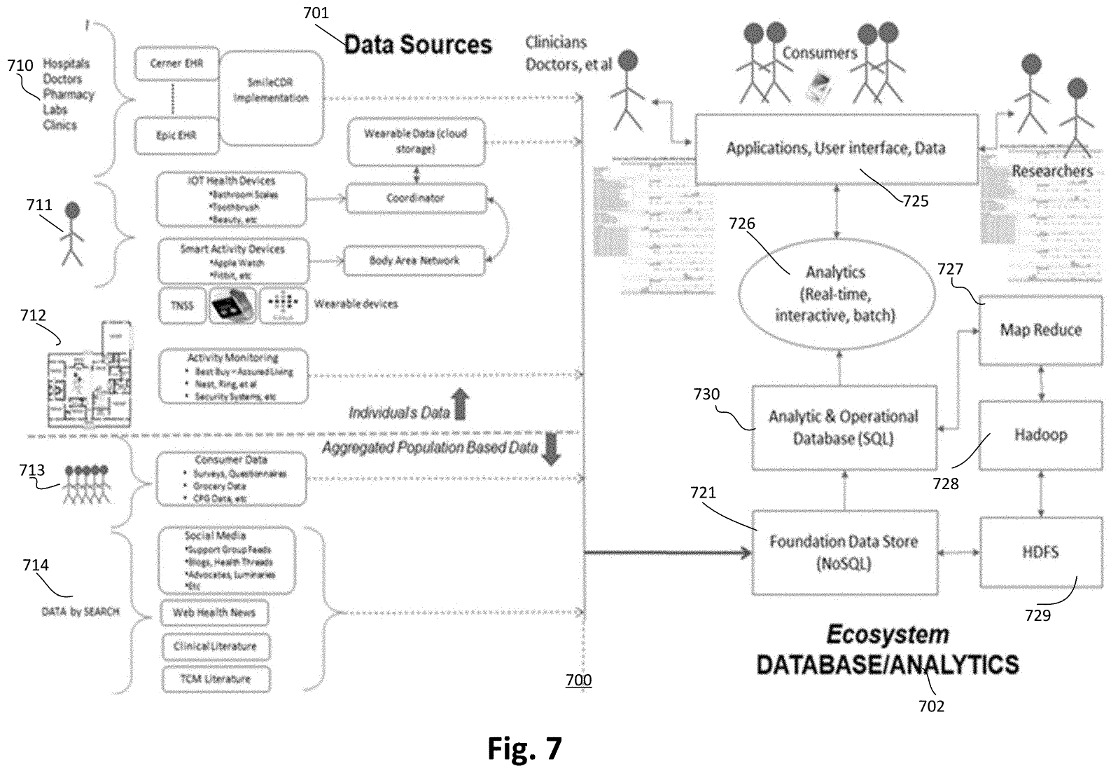

FIG. 7 illustrates an overall system 700 that combines data sources and performs data analysis in accordance to example inventions. System 700 includes a plurality of data sources 701 and a database/analytics 702 functionality. Data sources 701 includes the accumulation of many different data types, all representing data focused about the health parameters of individuals or groups of individuals. The various data types include traditional electronic health records (EHR/MHR) 710 that capture individual health records including doctor/hospital visits, laboratory results, imaging results, medications, insurance records, etc. The data includes whole classes of Internet of Things (“IOT”) devices 711, such as activity monitoring devices, communication devices, wearable devices incorporating sensors and analytics (e.g. the Apple Watch), home monitoring devices (e.g., the “Nest” device), a topical nerve stimulator/sensor (TNSS) family of devices such as patch 124 disclosed above. Data sources 701 include consumer generated data 713 including surveys and social media such as blogs, forums, etc. Data sources 701 further include search data 714 include specific medical databases (e.g., “PubMed”, clinical trials, regulatory records, etc.), medical references (e.g., Gray's Anatomy, etc.) and traditional Chinese medicine (“TCM”) sources.

The data is stored in various different data structures 702 and the data from various sources are represented in a Foundation Data Store 721. The various data types includes both structured data (e.g., EHR, consumer surveys) and real-time signals (e.g., EKG, activity tracking data, etc.).

In general, examples include two forms of data, those that have been preprocessed and distilled by their sources (e.g., “PubMed” articles, Clinical Trials, etc.) and those that have been minimally processed, or just raw data, and represent the original data captured (e.g., Fitbit data, EEG recordings, PSG data, etc.). Further, there are those data sources that are mixed, having elements of preprocessed data and minimally altered raw data (e.g., EHR). Another dimension is whether the data source originates from many individuals, or from a single individual (e.g., Clinical Trials vs Gray's Anatomy reference online book). Various data storage formats 727-730 are fed into analytics processing 726 to generate data for applications, a user interface, and additional data at 725.

In one example, correlation engine 380 unifies these forms of data by treating each as examples of “events” or “snapshots”. For example, data that is an EHR represents primarily single event data, such as a doctor visit, a doctor diagnosis, laboratory results or prescribed medications. This event data is characterized by a timestamp (e.g., day of visit, date/time of laboratory tests, etc.). Data from wearable devices are a series of “events” where that series may be hundreds or thousands of sequential events. In a similar manner, each event in the series can be characterized by an individual timestamp.

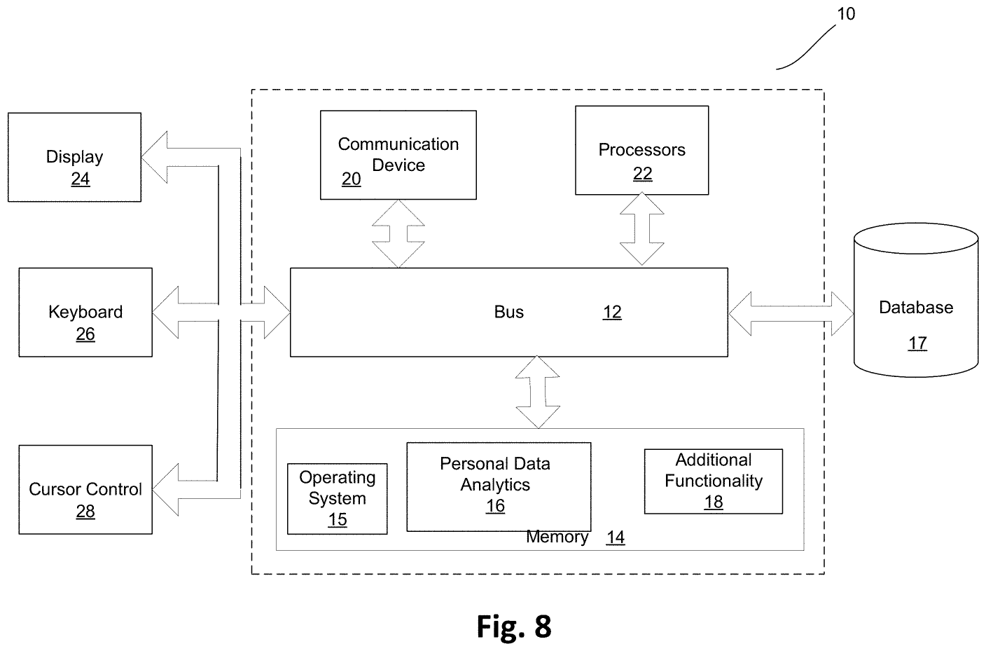

FIG. 8 is a block diagram of a computer server/system 10 in accordance with examples of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. For example, for functionality of a server, system 10 may need to include a processor and memory, but may not include one or more of the other components shown in FIG. 8, such as a keyboard or display. All or portions of system 10 may be used to implement any or all of the components shown in FIGS. 1-4 and 7 in some examples.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication device 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a personal data analytics module 16 that provides personal data analytics functionality, and all other functionality disclosed herein. System 10 can be part of a larger system. Therefore, system 10 can include one or more additional functional modules 18 to include the additional functionality. A database 17 is coupled to bus 12 to provide centralized storage for modules 16 and 18. In one embodiment, database 17 is a relational database management system (“RDBMS”) that can use Structured Query Language (“SQL”) to manage the stored data.

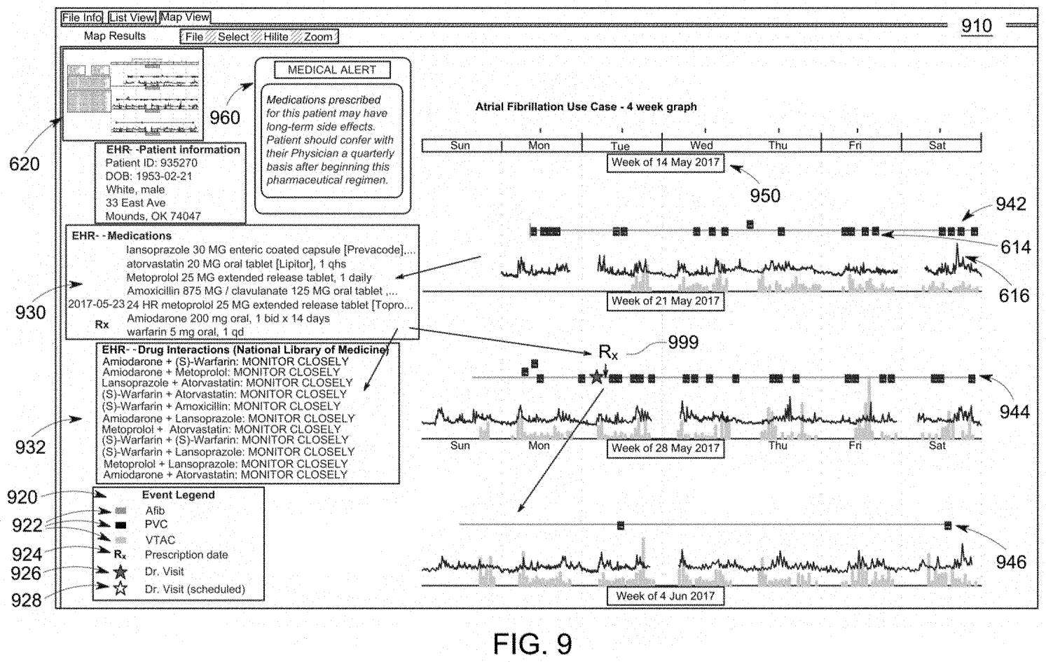

FIG. 9 is a UI 910 for displaying generated information, including correlations and alerts, in accordance to example inventions. FIG. 9 illustrates how example inventions combine data sources to produce new insights that cause alerts that can change a user's or clinician's behavior. In the example of FIG. 9, traditional patient EHR data such as doctor's visits and medication lists can be combined with wearable data such as Atrial fibrillation (“AFib”) events and EKG signals (measured and recorded by one or more wearables) to develop insights as to appropriate medical interventions.

In FIG. 9, a legend 920 is included to assist in interpretation of the cardiac-related events in the user interface. Legend 920 includes unique icons for cardiac events 922, prescriptions written 924, doctor visits 926 and scheduled doctor visits 928. Additional icons are added to legend 920 according to the content of the user interface.

Multiple biometrics are plotted on a timeline 950 in UI 910. The scale on timeline 950 is adjustable, to show a span of time varying, for example, from weeks to months to years. UI 910 includes navigation window 620, and line graph trace 616. In this example, event marker icons 614 show the series of atrial fibrillation events across week one 942, week two 944, and week three 946. Measurement of atrial fibrillation events during week one 942 resulted in the prescription of medications 930, which is displayed on a common timeline with the AFib events and the biometric data at 999. When the user began to take the medications, the frequency of atrial fibrillations was reduced, as shown in week three 946. Medication interaction analysis 932, through access to the National Library of Medicine drug interaction database, and other means, is included in UI 910. Medication interaction analysis 932, performed by example inventions, has triggered in the system an alert message 960. Alert message 960 causes a behavior change for the user in scheduling repeated reviews of their prescribed medications, and for the physician in maintaining awareness of possible side effects.

The data representing AFib could represent multiple patients and not just a single individual. This would be more representative of a clinical study where the medication is a constant variable and the observed reaction of a cohort of patients is the desired result.

For prescribed medications that should be ingested on a set schedule, the events of taking each medication on a set schedule can be plotted. In one example, the consuming of the medication may be assumed to have taken place on the schedule prescribed by the physician if there is no mechanism for detecting that actual ingesting of the medication. In another example, a system detects or records the taking of the medication, or for physician/nurse administered medicine those events are known, and the events are placed on the timeline. In both examples, correlations may be found between the events of taking medication and the biometrics plotted on the same timeline.

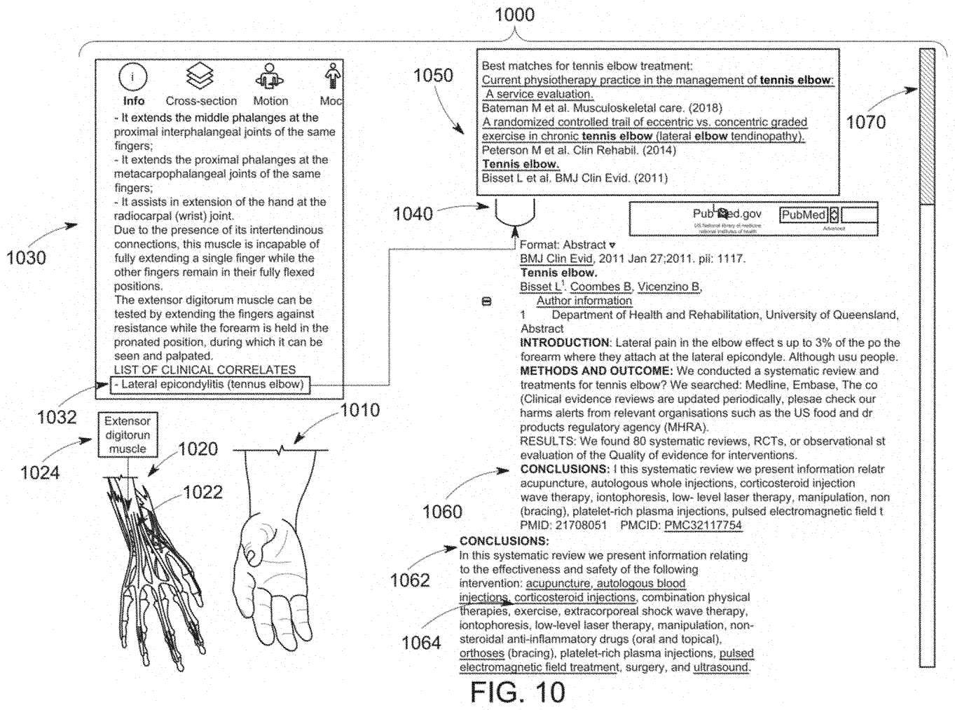

FIG. 10 is a UI 1000 for displaying generated information in accordance to example inventions. FIG. 10 shows an example of integration and coordination of information from a variety of disparate data sources into a cohesive medical condition user interface 1000, in this case for lateral epicondylitis. A surface image 1010 is provided, with an anatomical image 1020, including a target tissue image 1022, and a target tissue label 1024, to set the context for the viewer, which may be a user or a medical professional. A target tissue description 1030 is provided by extraction from a medical information database (in this example, “Complete Anatomy”, by 3D4Medical, Ltd.), this description including a list of medical conditions 1032 in the target tissue, such as Tennis Elbow. The medical condition is used as a parameter to extract from a research database 1040, such as “Pubmed”, a publication list 1050 pertaining to the medical condition. The publication list is presented in the same user interface with the aforementioned details. When the user selects a publication from publication list 1050, an abstract 1060 for the selected publication is presented on the same user interface (e.g., in a popup window). The conclusion 1062 is copied from abstract 1060 to facilitate the user's selection of one or more treatment 1064, several of which are highlighted as hyperlinks. Scroll bars 1070, or other means, are provided to view content which exceeds the size of the UI 1000 display space.

The user's selection of a treatment 1064 from UI 1000, such as that shown in FIG. 10, may in turn present new surface images 1010 or anatomical images 1020, or both.

As disclosed, combining activity data with EHR data can influence medications and/or dosage levels, similar to a drug interaction database that now includes the addition of the analysis of an individual's activity to check against appropriate drug prescriptions. For example, an excessive heart rate during mild exercise may indicate that the dosage level of the individual's heart medication may need to be adjusted. As another example, patch 124 may detect an unsafe increase in urinary urgency following the new prescription of a diuretic drug, which leads to a recommendation that the drug needs to be discontinued.

Further, placing hyperlinked clinical references on anatomical images representing various medical indications allows clinicians to provide better diagnostic capabilities. Further, combining clinical data with patient data can provide an assessment of the commercial viability of new medical interventions. For example, in the search for new drugs, devices, or interventions, example inventions provide insights into breakthrough clinical results. This will inform business decisions, research funding, and the future paths that clinicians will pursue in their work. Further, embodiments may use the data to classify a user's medical concerns/conditions in order to initiate some type of advertising to the user based on the conditions.

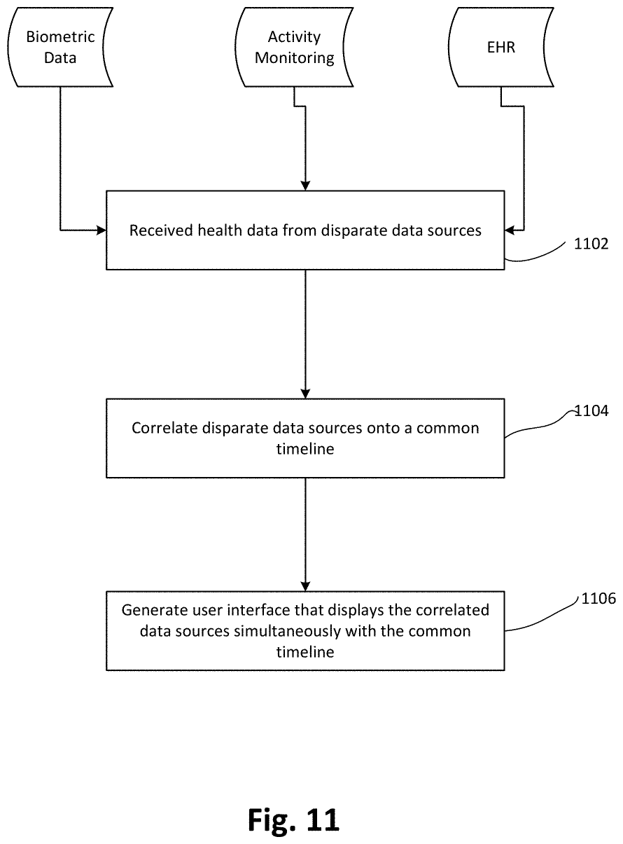

FIG. 11 is a flow diagram of the personal data analytics module 16 of FIG. 8 when generating personal data analytics in accordance to example embodiments. In one example, the functionality of the flow diagram of FIG. 11 is implemented by software stored in memory or other computer readable or tangible medium, and executed by a processor. In other examples, the functionality may be performed by hardware, for example through the use of an application specific integrated circuit (“ASIC”), a programmable gate array (“PGA”), a field programmable gate array (“FPGA”), etc., or any combination of hardware and software.

At 1102, health data is received from disparate data sources, including two or more of biometric data, activity monitoring, or an electronic health record.

At 1104, the disparate data sources are correlated onto a common timeline.

At 1106, a user interface is generated that displays the correlated data sources simultaneously with the common timeline.

Several example inventions are specifically illustrated and/or described herein. However, it will be appreciated that modifications and variations of the disclosed example inventions are covered by the above teachings and within the purview of the appended claims without departing from the spirit and intended scope of the invention.

What is claimed is:

1. A method of evaluating a health of a user, the method comprising:
receiving, from a plurality of disparate data sources, health data corresponding to the user, the plurality of disparate data sources comprising two or more of biometric data, activity monitoring, or an electronic health record;
correlating the disparate data sources onto a common timeline; and
generating a user interface that displays the correlated data sources simultaneously with the common timeline.

2. The method of claim 1, the correlating comprising generating events from the data sources and generating a corresponding timestamp for each event, the electronic health record generating one or more single events and the activity monitoring generating a series of events.

3. The method of claim 1, further comprising generating events from the data sources, the user interface comprising an icon on the common timeline corresponding to single events, a continuous line graph on the common timeline corresponding to continuous events, and a bar chart on the common timeline corresponding to discrete data.

4. The method of claim 1, further comprising one or more alerts in response to the correlating.

5. The method of claim 2, the electronic health record comprising doctor visits, medicine prescribed, medical images and laboratory tests.

6. The method of claim 1, the activity monitoring generated by a smart patch comprising:
a flexible substrate comprising adhesive on a first side adapted to adhere to a dermis of the user;
an electronic package directly coupled to the substrate, the electronic package comprising a control unit and one or more stimulators; and
electrodes directly coupled to the substrate and the electronic package and disposed between the adhesive and the dermis.

7. The method of claim 1, the biometric data comprising one or more of a step count, pulse rate, blood pressure, respiration rate, hydration, blood sugar levels or body mass index.

8. The method of claim 4, the alerts comprising a revision of medication levels.

9. The method of claim 1, the correlation further comprising a determination of a medical condition.

10. The method of claim 9, the user interface further comprising displaying an anatomical image that corresponds to the medical condition.

11. A non-transitory computer-readable medium storing instructions which, when executed by at least one of a plurality of processors, cause the processor to evaluate a health of a user, the evaluating comprising:
receiving, from a plurality of disparate data sources, health data corresponding to the user, the plurality of disparate data sources comprising two or more of biometric data, activity monitoring, or an electronic health record;
correlating the disparate data sources onto a common timeline; and
generating a user interface that displays the correlated data sources simultaneously with the common timeline.

12. The computer-readable medium of claim 11, the correlating comprising generating events from the data sources and generating a corresponding timestamp for each event, the electronic health record generating one or more single events and the activity monitoring generating a series of events.

13. The computer-readable medium of claim 11, the evaluating further comprising generating events from the data sources, the user interface comprising an icon on the common timeline corresponding to single events, a continuous line graph on the common timeline corresponding to continuous events, and a bar chart on the common timeline corresponding to discrete data.

14. The computer-readable medium of claim 11, the evaluating further comprising one or more alerts in response to the correlating.

15. The computer-readable medium of claim 14, the electronic health record comprising doctor visits, medicine prescribed, medical images and laboratory tests.

16. A personal data analytics system comprising:
a plurality of disparate data sources configured to provide health data corresponding to the user, the plurality of disparate data sources comprising two or more of biometric data, activity monitoring, or an electronic health record;
a correlation engine coupled to the plurality of data sources and configured to correlate the disparate data sources onto a common timeline; and
a user interface configured to display the correlated data sources simultaneously with the common timeline.

17. The personal data analytics system of claim 16, the correlate comprising generating events from the data sources and generating a corresponding timestamp for each event, the electronic health record generating one or more single events and the activity monitoring generating a series of events.

18. The personal data analytics system of claim 16, the correlation engine further configured to generate events from the data sources, the user interface comprising an icon on the common timeline corresponding to single events, a continuous line graph on the common timeline corresponding to continuous events, and a bar chart on the common timeline corresponding to discrete data.

19. The personal data analytics system of claim 16, the activity monitoring generated by a smart patch comprising:
a flexible substrate comprising adhesive on a first side adapted to adhere to a dermis of the user;
an electronic package directly coupled to the substrate, the electronic package comprising a control unit and one or more stimulators; and
electrodes directly coupled to the substrate and the electronic package and disposed between the adhesive and the dermis.

20. The personal data analytics system of claim 16, the biometric data comprising one or more of a step count, pulse rate, blood pressure, respiration rate, hydration, blood sugar levels or body mass index.
